(19) United States
(12) Patent Application Publication
(10) Pub. No.: US 2007/0065503 A1
(43) Pub. Date: Mar. 22, 2007

(54) Active substance-doped absorbing polymer particles, composition comprising polycondensate matrix and absorbant polymer for release of a wound treatment substance

(75) Inventors: Jorg Harren, Krefeld (DE); Ursula Nielinger, Krefeld (DE); Dieter Oppenberg, Muhlheim/Ruhr (DE); Harald Schmidt, Tonisvorst (DE)

Correspondence Address: SMITH MOORE LLP, P.O. BOX 21927, GREENSBORO, NC, 27420, US

(73) Assignee: STOCKHAUSEN GMBH, Krefeld (DE)

(21) Appl. No.: 10/563,747

(22) PCT Filed: Jul. 1, 2004
(86) PCT No.: PCT/EP04/07140

§ 371 Date: Apr. 19, 2006

(30) Foreign Application Priority Data
Jul. 8, 2003 (DE) 103 30 960.8

(51) Int. Cl. C08F 8/30; A61K 9/66

(52) U.S. Cl. 424/455; 524/556; 525/126

(57) ABSTRACT

A water-absorbing composition, a process for producing a water-absorbing composition, a composite comprising a water-absorbing composition, and the use of a water-absorbing composition for releasing an active substance (e.g., a wound treatment substance, a care substance, or combinations thereof) are disclosed. A water-absorbing composition can include active substance-doped water-absorbing polymer particles comprising: Φ1.) an active substance in a quantity in the range from about 0.001 to about 30 wt. %; and Φ2.) an absorber matrix in a quantity in the range from about 70 to about 99.999 wt. %. Also, a hygiene article and a wound treatment means, including a water-absorbing composition, are disclosed.

This application is a national stage application under 35 U.S.C. 371 of international application No. PCT/EP2004/007140 filed Jul. 1, 2004, which is based on German Application No. DE 103 30 960.8 filed Jul. 8, 2003, and claims priority thereto.

The present invention concerns active substance-doped water-absorbing polymer particles, a water-absorbing composition, a process for producing a water-absorbing composition, a water-absorbing composition obtainable by this process, a composite comprising this water-absorbing composition, the use of this water-absorbing composition or of a water-absorbing polymer comprised in the water-absorbing composition for release of a wound treatment substance, the use of this water-absorbing composition or of a water-absorbing polymer comprised in the water-absorbing composition for producing a means for treating wounds, a process for treating wounds with this water-absorbent composition or of a water-absorbing polymer comprised in the water-absorbent composition as well as the use of this water-absorbent composition or of a water-absorbing polymer comprised in the water-absorbent composition in a hygiene article or wound treatment means.

A number of documents are known from the art, in which active substance-doped water-absorbing polymer particles are disclosed. Thus, US 2003/0004479 A1 describes a water-absorbent composition comprising a water-absorbing polymer and a plant powder as an active substance, whereby the water-absorbing polymer is post-crosslinked in the surface area. This composition can bind unpleasant odors because of the active substance contained therein.

DE 101 42 918 A1 discloses a self-sticking gel matrix based on unneutralized polyacrylate, which can be doped with active substances for cosmetic or pharmaceutical treatment of the skin or systemic administration of medicaments. Since the gel matrix is applied as a connected layer on a substrate, the release of active substance is thereby limited.

DE 102 57 002 A1 discloses a skin care means comprising foam hydrogels based on crosslinked, acid groups-comprising foam polymers. The polymers, which are provided as flat structures, comprise a skin care agent and are used in hygiene articles. The gel matrix cannot be incorporated into customary hygiene articles and/or wound dressings provided for water-absorbing polymer particles without significant and thereby costly changes to the construction.

A number of documents are further known from the art that disclose the combination of water-absorbing polymers and polyurethanes in a matrix and their use in wound dressings and wound plasters.

Thus, EP 0 196 364 A2 discloses hydrophilic polyurethane foams which are filled with water-absorbing polymers based on an acrylic acid-potassium salt polymer and can be used for medicinal purposes.

Further, DE 42 33 289 A 1 teaches hydrophilic polyurethane gel foams with water-absorbing polymers, which can be used in medicine.

Thus in general the embodiments according to the invention include at least attenuating the disadvantages arising from the state of the art or to overcome them completely.

In particular, an embodiment according to the present invention is to make available a water-absorbent composition, or an agent comprising this water-absorbent composition, which can care for the skin and increase the resistance of the skin. This is particularly the case for areas where wounds arise. These wounds occur in wearers of diapers by long-term contact with excrements and/or on lying down for long periods. Furthermore, a strengthening of the resistance of the skin in bed-ridden patients wearing incontinence articles is advantageous, since in this way the danger of the presence of wounds is prevented.

In addition, an embodiment of the invention can be considered to be caring for the skin or accelerating healing of wounds or respectively to make available a hereto suited water-absorbent composition or a means comprising this water-absorbing composition.

Further, an embodiment of the invention includes increasing the wearing comfort of wound dressings, of skin plasters, in particular wound plasters or hygiene articles and in particular to prevent the displacement and slipping of particulate water-absorbing polymers in wound dressings, in skin plasters, in particular in wound plasters or in hygiene articles or components thereof aimed at liquid absorption without the care and treatment properties of the skin or wound plaster, thereby being disadvantageously influenced.

In addition, an embodiment of the invention lies in designing the liquid-management in wound dressings, in skin plasters, in particular in wound plasters, or in hygiene articles, in such a way that the wound dressings, skin plasters, or hygiene articles can be used over as long a time period as possible, without resulting in moisture- or even wet-blockages and the respective care, wound prevention, or healing effect of the wound dressings, skin plasters, or hygiene articles being negatively influenced.

Further, an embodiment of the invention includes making available an active substance, in one aspect a wound treatment substance or a care substance, which is worked into a carrier matrix in the formation of this carrier matrix, and is less significantly reduced by the destruction of the active substance due to its incorporation. The danger of reducing the effectiveness by destroying the active substance exists in particular if this is chemically changed by the compounds used in producing the matrix. This danger exists in particular if the matrix comprises a polymer which is based on monomers with functional groups which can react with functional groups of the active substance, whereby the effectiveness or availability of the active substance is reduced. This can be observed in particular in the incorporation of active substances in a polycondensate matrix, whereby above all the isocyanate groups react with the functional groups of the active substance.

In addition, an embodiment of the invention includes providing hygiene articles which prevent the formation of wounds and skin sores caused in particular by bed sores or unsuitable nutrition, or to alleviate these and promote their healing, together with high wearer comfort and absorption performance. A further embodiment according to the invention includes making available hygiene articles which can care for skin which is in contact with the hygiene article, together with high wearer comfort and absorbent performances.

The above embodiments are the subject matter of claims and detailed in the following.

An embodiment of the invention relates to active-doped water-absorbing polymer particles comprising:
Φ1. as active substance, a care substance, in particular a skin care substance, or a wound treatment substance, or salts thereof, in one aspect in a quantity in the range from about 0.001 to about 30 wt. %, in another aspect from about 0.1 to about 20 wt. %, and in yet another aspect from about 1 to about 15 wt. %, respectively based on the active substance-doped water-absorbing polymer particles,

Φ2. an absorber matrix in one aspect in a quantity in the range from about 70 to about 99.999 wt. %, in another aspect from about 80 to about 99.9 wt. % and in yet another aspect from about 90 to about 99 wt. %, respectively based on the active substance-doped water-absorbing polymer particles,

wherein the absorber matrix comprises at least about 90 wt. %, based on the absorber matrix, a crosslinked polyacrylic acid,

wherein the crosslinked polyacrylic acid comprises at least about 90 wt. %, based on the crosslinked polyacrylic acid, an acrylic acid which is partially neutralized to at least about 30 mol. %.

In addition, in an embodiment of the invention relating to the active substance-water doped water-absorbing polymer particles, the active substance is distributed in one aspect over at least about 70, in another aspect over at least about 80, in yet another aspect over at least about 90 vol. %, and in even yet another aspect over the entire absorber matrix. In this case, the active substance can be distributed homogenously over the entire absorber matrix.

Furthermore in another embodiment, the active substance-doped water-absorbing polymer particles have a residual monomer content of the monomer on which the water-absorbing polymer particle is based, in one aspect under about 500 ppm, in another aspect under about 300 ppm, in yet another aspect under about 200 ppm, and in even yet another aspect under about 146 ppm, respectively based on the water-absorbing polymer particles. The residual monomer content is determined according to European nonwoven and diaper association. Recommended Test 410.1-99 (ERT 410.1-99).

Further, in yet another embodiment, the active substance-doped water-absorbing polymer particles have an active substance availability in one aspect of at least about 40 wt. %, in another aspect at least about 70 wt. %, in yet another aspect at least about 85 wt. % and in even yet another aspect at least about 95 wt. % of an active substance present in the water-absorbing polymer particles, determined according to the herein-described extraction test.

In a further embodiment according to the invention, the active substance-doped water-absorbing polymer particles have a particle size distribution, whereby at least about 80 wt. % of the particles have a particle size determined according to ERT 420.1-99 in one aspect in a range from about 20 μm to about 900 μm, in another aspect in the range from about 150 μm to about 600 μm, and in yet another aspect in the range from about 200 μm to about 400 μm.

In an additional embodiment according the invention, the active substance-doped water-absorbing polymer particles according to one aspect comprise less than about 15 wt. %, in another aspect less than about 10 wt. %, and in yet another aspect less than about 5 wt. %, based on the active substance-doped water-absorbing polymer particles, of particles with a particle size in one aspect of about <100 μm, in another aspect about <130 μm, and in yet another aspect about <150 μm.

In a further embodiment according to the invention, the active substance-doped water-absorbing polymer particles comprise in one aspect less than about 15 wt. %, in another aspect less than about 10 wt. %, and in yet another aspect less than about 5 wt. %, based on the active substance-doped water-absorbing polymer particles, of particles with a particle size in one aspect of about >950 μm, in another aspect about >850 μm, and in yet, another aspect about >650 μm.

The above particle size distributions and particle sizes are particularly advantageous for an even delivery and distribution of the active substance and for good wearer comfort. In addition, it has been shown that the above particle size distributions and particle sizes can be incorporated particularly well into flexible matrices, which, if incorporated into plasters or wound dressings, increase the adaptability of these plasters or wound dressings to the skin, in particular in wound dressings to the form of the wound and to the movements thereof.

An active substance includes a care substance such as skin care substances that
a) clean the skin,
b) perfume the skin,
c) change the appearance of the skin,
d) protect the skin, or
e) maintain the skin in good condition.

In an embodiment, an active substance comprises a care substance including an agent that has in one aspect at least one, in another aspect at least two, and in yet another aspect at least three of the above-mentioned properties a) to e). In other embodiments of the invention, active substance-doped water-absorbing polymer particles further include care substances characterized by the following properties or respective property combinations: a, b, c, d, e, ab, ac, ad, ae, bc, bd, be, cd, ce, de, abc, abd, abe, acd, ace, ade, bcd, bce, bde, cde, abcd, abce, acde, bcde, or abcde.

In one aspect, plant extracts are used as care substances. In another aspect, substances that have no deodorizing effect are used as care substances.

In yet another aspect of the invention, the care substance or at least one substance of the mixture of care substances has as a functional group a double bond, an OH group, an NH group, or a COOH group, or a salt of at least one of these groups, beneficially an OH group.

In even yet another aspect, care substances are selected from 18-β-glycyrrhetinic acid from liquorice root extract (Glycyrrhiza glabra), such as for example in a purity of greater than about 99 % pure substance in the extract, Aescin in horse chestnut (Aesculus hippocastanum), allantoin, aloe vera (comprising principally sugar, anthraquinone, and minerals such as zinc), amino acids such as for example alanine, arginine, serine, lysine, ammonium glycyrrhizate from liquorice root extract, in an aspect in a purity of almost about 100 % pure substance in the extract, apigenine from camomile extract (matricaria recutita), Arnica, in particular Arnica montana or arnica chamissonis, asiaticosides and madecassosides in the centella asiatica extract, avena anthramides from oat extract (avena sativa), avocado oil, azulene from camomile extract (matricaria recutita), biotin (vitamin H), bisabolol from camomile extract (matricaria recutita), brown algae extract (ascophyllum nodosum), chlorogenic acid in water extract of Japanese honeysuckle (lonicera japonica), coenzyme-Q10, creatine, dexpanthenol, disodium glycyrrhizate from liquorice root extract, in an aspect in a purity of almost about 100 % pure substance in the extract, extract of red algae (asparagopsis armata), flavonoids from birch extract (betula alba), flavonoids, vitexine in extract of passion flower (passiflora incarnata), flavonoids, vitexine in linden-tree extract (tilia platyphyllos), ginkgo flavone glucosides and terpene lactones in ginkgo extract (ginkgo biloba), ginsenosides in Ginseng extract (panax ginseng), glycogen, grapefruit extract, hamamelis extract from Virginian witch hazel (hamamelis virgiana), honey, isoflavone glucosides in sorrel extract (trifolium pratense), Saint John's wort extract from Saint John's wort (hypericum perforatum), jojoba oil, lecithin, maize oil (zea mays), evening primrose oil, niacinamide, Oenotheine B in rosebay willow-herb extract (epilobium angustifolium), oleurepein in olive extract (olea europea), phytocohesin (sodium-beta-sitosterol sufate, plankton extract (tetraselmis suecica, spirulina and others), polyphenols, catechols from grape seed extract (vitis vinifera), polyphenols, catechols from green tea (camellia sinensis), marigold extract (calendula officinalis), rosemary acid in melissa extract (melissa officinalis), sea buckthorn oil, β-glucans from oats (avena sativa), stearyl glycyrrhetenic acid (stearyl ester of 18-β-glycyrrhetinic acid), sterols, sitosterol in nettle extract (urtica dioca), sweet almond oil (prunus dulcis), vitamin C and its esters, vitamin E and its esters, wheat germ oil, zinc gluconate/magnesium aspartate/copper gluconate, zinc sulfate, or zinc oxide.

In one aspect, the active substance-doped water-absorbing polymer particles according to an embodiment of the invention comprise as care substance a mixture of at least two of the above mentioned care substances. In another aspect, the active substance-doped water-absorbing polymer particles according to an embodiment of the invention, comprise as a care substance a mixture of at least 3 of the above mentioned care substances.

Wound treatment substances are advantageously understood as those pharmaceutical active substances with wound-healing property, which fall under the term of pharmaceuticals of § 2, Arzneimittelgesetz in the version from 11 Dec. 1998, whereby the wound-healing active substances given in the red list of the year 2002 are advantageous.

A “wound-treating substance” in the sense of this invention is a substance or a mixture of at least two substances which can as such or in combination with further active components advance the process of wound-healing, whether it is for example by a disinfection effect in the wound area, by a promotion of homeostasis of the wound environment, by a stimulation of the cell growth in the wound area, or by a stimulation of the secretion of, for example, proteins such as collagen or elastin, of proteoglucanes or of messenger substances by the skin cells in the wound area.

Advantageously, the “wound-treatment substances” according to an embodiment of the invention have, as functional groups, a double bond, an OH group, an NH group, or a COOH group, or a salt of at least one of these groups, beneficially an OH group. In an aspect, the wound-treatment substance has about 2 to about 100 carbon atoms and about 1 to about 20 oxygen atoms. The above properties are likewise applicable for the active substances or pharmaceutically active substances according to an embodiment of the invention.

In general, as wound-treatment substances based on plant extracts, Equisetum arvense, Aloe barbadensis, Arnica montana, Arnica chamissonis, Symphytum officinale, Solanum dulcamara, Echinacea pallida, Potentilla erecta, Trigonella foenum-graecum, Juglans regia, Linum usitatissimum, Terminalia sericea, Oenothera biennis, Centella asiatica, Arctium lappa, Capsella bursa-pastoris, Hypericum perforatum, Matricaria recutita, Chamomille recutita, Agrimonia eupatoria, Centaurea cyanus, Larrea tridentata, Populus spec., Echinacea pupurea, Calendula officinalis, Aesculus hippocastanum, Salvia officinalis, Plantago lanceolata, Quercus robur, Glycyrhiza glabra, Quercus petraea, Hamamelis virgian, Cardiospermum halicacabum, Betula, Urtica dioica, Buxus chinensis, Lavandula angustifolia, Lavandula hybrida, Crocus sativus, Smilax aspera, Melaleuca altemifolia, amino acids or viola tricolor, or salts thereof, or derivatives, or mixtures, or at least two thereof are used according to an embodiment of the invention.

Furthermore, as further wound-treatment substances are considered according to an embodiment of the invention, vitamins and the like, as well as glucosamine sulfate allantoin, biotin, chondroitin sulphate, Coenzyme Q10, dexpanthenol, honey/honey-extract, niacin amide, propolis, vitamin A or its esters, vitamin C and its esters, vitamin E and its esters, or salts thereof, or derivatives, or mixtures of at least two thereof.

According to an aspect of the invention, the wound-treatment substances are dexpanthenol or extracts of marigold, another aspect calendula oil; of hamamelis, beneficially D-hamelose; or of camomile, yet another aspect the oil of camomile flowers—and still yet another aspect bisbolol or azulene—or mixtures of at least two of the above substances.

In an embodiment, respectively, one of the above wound-treatment substances can be present in a mixture as a main component in an embodiment of this invention, whereby this main component can be present in one aspect to at least about 50 wt. %, in another aspect to at least about 70 wt. %, and in yet another aspect to at least about 95 wt. %, respectively based on the mixture.

A comparison of the list of aspects of care substances with a list of the aspects of wound-treatment substances shows, as active substance, compounds or compositions can also be used that have a wound-healing effect as well as a caring effect, whereby active substances of this type are aspects according to an embodiment of the invention. Examples of these active substances include, for example, allantonin, recutita, arnica, in particular arnica montana or arnica chamissonis, biotin, coenzyme Q10, dexpanthenol, honey or honey extract, amino acid, niacinamides, vitamin C or its esters or vitamin E, or its esters.

In a further aspect, a mixture of a wound-healing and of a caring substance is used as an active substance. In addition, the active substances given according to an embodiment of the invention comprise also the corresponding active salts thereof.

The active substance-doped water-absorbing polymer particles are used in one aspect in hygiene articles, for example in diapers or sanitary napkins.

Further, another embodiment of the present invention relates to a water-absorbing composition comprising:
Γ1 a polycondensate matrix, based on at least one polycondensate monomer with at least one polycondensate group, and
Γ2 a particulate water-absorbing polymer comprising an active substance, in an aspect a wound-treating substance or a skin care substance, or salt thereof, with at least one functional group that can react with the polycondensate group to form a covalent bond, or an active substance-doped water-absorbing polymer particle according to an embodiment of the invention,
   whereby the particulate water-absorbing polymer or the active substance-doped water-absorbing polymer particles or both is at least partially surrounded by the polycondensate matrix,
   wherein at least the particulate water-absorbing polymer or the active substance-doped water-absorbing polymer particle or both comprise the wound-treating substance or the active substance or both in one aspect to at least about 60, in another aspect to at least about 80, and in yet another aspect to at least about 90 wt. %, respectively based on the quantity contained in the composition, and
   wherein the water-absorbing composition has in one aspect an active substance availability of at least about 10 wt. %, in another aspect at least about 30 wt. %, in yet another aspect at least about 70 wt. %, in yet another aspect at least about 80 wt. %, and in even yet another aspect at least about 90 wt. % of the active substance contained in the water-absorbing composition, according to the herein given extraction test.

In one embodiment of the invention relating to a composition, Γ2 comprises an active substance doped water-absorbing polymer particle according to an embodiment of the invention.

In another embodiment of the invention relating to a composition, Γ2 comprises a particulate water-absorbing polymer comprising an active substance, in an aspect a wound-treatment substance or a skin care substance, or salts thereof, with at least one functional group, which can react with the polycondensate group by forming a covalent linkage.

By “water-absorbing” is understood, according to an embodiment of the invention, besides the capacity of a material to absorb water—in one aspect at least about 4 times, in another aspect at least about 10 times, and in yet another aspect at least about 100 times its own weight—to form a hydrogel, any absorption of aqueous liquids, in particular aqueous body fluids such as urine, blood, and blood components such as matter, lymph fluids or blood serum.

As wound-treatment substances and care substances are considered advantageously those wound-treatment substances and care substances or mixtures of wound-treatment substances and care substances that were mentioned in the context of the active substance-doped water-absorbing polymer according to an embodiment of the invention as beneficial wound-treatment substances and care substances.

According to an embodiment of the invention, a water-absorbing composition comprises as composition components:
a. a polycondensate matrix in a quantity in one aspect in the range from about 5 wt. % to about 98.999 wt. %, in another aspect in the range from about 20 wt. % to about 95.9 wt. %, and in yet another aspect in the range from about 30 wt. % to about 93.3 wt. %, respectively based on that water-absorbing composition;
b. a particulate water-absorbing polymer, in a quantity in one aspect in the range from about 1 wt. % to about 70 wt. %, in another aspect in the range from about 3 wt. % to about 25 wt. %, and in yet another aspect in the range from about 5 wt. % to about 15 wt. %, respectively based on the water-absorbing composition;
c. an active substance, in an aspect a wound-treating substance or a skin care substance, comprised in the particulate water-absorbing polymer, in a quantity in one aspect in the range from about 0.001 wt. % to about 25 wt. %, in another aspect in the range from about 0.001 wt. % to about 20 wt. %, and in yet another aspect in the range from about 0.1 wt. % to about 15 wt. %, respectively based on the water-absorbing composition; as well as
d. one or more additives, in a quantity in one aspect in the range from 0 wt. % to about 50 wt. %, in another aspect in the range from about 0.1 wt. % to about 40 wt. %, and in yet another aspect in the range from about 1 wt. % to about 30 wt. %, respectively based on the water-absorbing composition;
   wherein the sum of the weight quantities of the composition components a to d gives about 100 wt. %. The composition of this embodiment is used in one aspect in wound dressings and skin plasters, and in another aspect in wound plasters.

The following concentration and weight part specifications for skin care agents are likewise valid in embodiments of this invention relating to active substances.

Furthermore, the invention relates to a process for producing a water-absorbing composition, wherein a particulate water-absorbing polymer comprising an active substance, in an aspect a wound treatment substance or a care substance (e.g., skin care substance), is at least partially incorporated into a polycondensate matrix based on at least one polycondensate monomer, wherein the particulate water-absorbing polymer comprising the active substance or an active substance-doped water-absorbing polymer particle according to an embodiment of the invention or mixtures thereof are brought into contact with the polycondensate monomer before completion of the polycondensate matrix formation.

In a process according to an embodiment of the invention, it is an aspect that the active substance be incorporated into the water-absorbing polymer comprising the active substance before completion of the formation of the water-absorbing polymer. According to an embodiment of the invention, it is understood that the formation of the water-absorbing polymers is substantially completed if the content in residual monomers on which the water-absorbing polymer is based lies in one aspect under about 500 ppm, in another aspect under about 300 ppm, in yet another aspect under about 200 ppm, and in even yet another aspect under about 146 ppm, respectively based on the water-absorbing polymer. The residual monomer content is determined according to ERT 410.1-99.

The low residual monomer content promotes wound healing and skin care and contributes to the improvement of the wearing comfort of skin plasters, in particular wound plasters or wound dressings, since tissue and in particular skin irritations are reduced or completely avoided, which can easily arise upon long-term wearing of the skin plaster or of the wound dressing.

It is further an aspect that the active substance is already added to the monomers before the start of the polymerization reaction for formation of the water-absorbing polymer, so that the active substance is incorporated into the water-absorbing polymer during the polymerization reaction.

The incorporation of the active substance into the water-absorbing polymer during the polymerization of the water-absorbing polymer can occur by means of all processes common to the skilled person. On the one hand, the active substance can be incorporated into the water-absorbing polymer via the solvent used in the production of the water-absorbing polymer. On the other hand, the active substance can be added to the monomer, oligomer, or pre-polymer, used for formation of the water-absorbing polymer, or at least two thereof. In both above variants, the active substance can be present as a solution, emulsion, or suspension. Further, both above variants can be combined with each other.

In another embodiment of the process according to the invention, the active substance is incorporated after completion of the formation of the water-absorbing polymer or during the further processing thereof or both. This incorporation can occur in a gel of the water-absorbing polymer. It is here beneficial that the gel comprises a water quantity based on the water-absorbing polymer in one aspect of about 0.2 to about 20 times, in another aspect about 1 to about 10 times, and in yet another aspect about 2 to about 4 times, in order to achieve as uniform as possible an incorporation of the active substance.

This can occur, on the one hand, by absorption of the active substance by means of a liquid, mostly aqueous carrier in which the active substance is dissolved. In the incorporation of the active substance during the further processing, it is beneficial that the active substance in a liquid, in an aspect a aqueous phase is incorporated into the water-absorbing polymer, optionally during the following described “post-crosslinking,” with which a surface crosslinking is achieved. In an aspect, this occurs together with the post-crosslinkers used for this purpose.

Combinations of the above process variants are also possible. In the incorporation of the active substance before completion of the formation of the water-absorbing polymer, a uniform doping of the water-absorbing polymer can be achieved. If the active substance is incorporated after completion of the formation of the water-absorbing polymer or during the further processing thereof or both, a doping of the water-absorbing polymer particles can then be achieved in the outer wall surface area thereof. A combination of both process variants leads generally to a polymer with a different concentration in the inner and outer region of the water-absorbing polymer particle whereby, in an aspect, the concentration of active substance can be mostly higher in the outer region.

The thus-obtained water-absorbing polymer doped with active substance, henceforth named “doped polymer,” can generally be incorporated into the polycondensate matrix in any way known to the skilled person. In an aspect, the doped polymer is incorporated into the polycondensate matrix before completion of the formation, i.e., before substantially all reactive functional groups of a polycondensate matrix monomer have reacted. This can occur by adding the doped polymer either to the solvent used in formation of the polycondensate matrix or to a polycondensate matrix monomer. In an aspect, the addition to a polycondensate matrix monomer can occur, whereby the addition to a polycondensate matrix monomer which is free from reactive functional groups, in one aspect free from reactive functional groups which can react with functional groups of the doped polymer, for example from a polyol in the case of the polyurethane matrix as polycondensate matrix.

The invention also relates to a process for producing an active substance-doped water-absorbing polymer particle, wherein a care substance or a wound-treatment substance is incorporated into an absorber matrix,

wherein the absorber matrix comprises at least about 90 wt. %, based on the absorber matrix, a crosslinked polyacrylic acid, and

wherein the crosslinked polyacrylic acid comprises at least about 90 wt. %, based on the crosslinked polyacrylic acid, an acrylic acid partially neutralized to at least about 30 mol. %.

As water-absorbing polymer, all polymers are aspects that have already been mentioned in the context of the process according to an embodiment of the invention for producing the water-absorbing composition. In an aspect, care substances and wound treatment substances are those that have been mentioned in the context of the active substance-doped polymer particles according to an embodiment of the invention.

The incorporation of the care substance or of the wound treatment substance into the absorber matrix can be incorporated, as described above in the context of the incorporation of the active substance into the water-absorbing polymer in the process according to an embodiment of the invention for producing a water-absorbing composition, during the polymerization reaction, and so before completion of the formation of the water-absorbing polymer particle, or after completion of the formation of the water-absorbing polymer or during the further processing thereof, or both. With respect to the type and method of carrying out the process, reference is made to the details in the context of the incorporation of the active substance into the water-absorbing polymer in the process according to an embodiment of the invention for producing the water-absorbing composition.

In an embodiment of the invention, the care substance for the wound-treatment substance is incorporated into the absorber matrix in such a quantity that the active substance-doped water-absorbing polymer particles generated are based on
Λ1. in one aspect about 0.001 wt. % to about 30 wt. %, in another aspect from about 0.1 wt. % to about 20 wt. %, and in yet another aspect from about 1 wt. % to about 15 wt. % care substance or wound-treatment substance and
Λ2. in one aspect about 80 wt. % to about 99.9 wt. % and in another aspect from about 90 wt. % to about 99 wt. % absorber matrix, wherein the sum of the components Λ1 and Λ2 amounts to about 100 wt. %.

In addition, the invention relates to a water-absorbing composition and active substance-doped water-absorbing polymer particles, obtainable according to the above process.

In the composition according to an embodiment of the invention and the active substance-doped water-absorbing polymer particles, the water-absorbing polymer has in an aspect at least one, and in another aspect all of the following properties:
A1) particle size distribution, whereby at least about 80 wt. % of the particles have a particle size in one aspect in a range from about 20 μm to about 900 μm, in another aspect in the range from about 150 μm to about 600 μm, and in yet another aspect in the range from about 200 μm to about 400 μm according to ERT 420.1-99;
A2) a Centrifuge Retention Capacity (CRC) in one aspect of at least about 10 g/g, in another aspect at least about 20 g/g, and in yet another aspect in a range from about 30 g/g to about 50 g/g according to ERT 441.1-99;
A3) an Absorption Against Pressure (AAP) at about 0.7 psi (4826 Pa) of, in one aspect, at least about 4 g/g, in another aspect at least about 6 g/g and in yet another aspect in a range from about 8 g/g to about 25 g/g according to ERT 442.1-99;
A4) a water-soluble polymer content after about 16 hours of extraction of, in one aspect, less than about 25 wt. %, in another aspect less than about 20 wt. % and in yet another aspect less than about 15 wt. %, respectively based on the total weight of the water-absorbing polymer, according to ERT 470.1-99,
A5) a residual moisture of, in one aspect, at most about 15 wt. %, in another aspect at most about 10 wt. % and in yet another aspect at most about 5 wt. %, respectively based on the total weight of the water-absorbing polymer, according to ERT 430.1-99.

Each of the combinations of features generated from the features A1 to A5 represent an embodiment of the invention, whereby the following feature combinations respectively represent aspects of embodiments: A1, A2, A1A2A3, A1A2A3A4, A1A3, A1A4, A1A3A4, A1A2A4, A2A3, A2A3A4, A2A4 as well as A3A4, whereby all of the above combinations with A1 are aspects.

In the water-absorbing composition and the active substance-doped water-absorbing polymer particles according to embodiments the invention, it is further an aspect that the water-absorbing polymer is based on
(α1) in one aspect about 0.1 wt. % to about 99.999 wt. %, in another aspect about 20 wt. % to about 98.99 wt. %, and in yet another aspect about 30 wt. % to about 98.95 wt. % of polymerized, ethylenically unsaturated, acid group-containing monomers or salts thereof or polymerized, ethylenically unsaturated monomers containing a protonated or quatemated nitrogen, or mixtures thereof, whereby mixtures comprising at least ethylenically unsaturated, acid group-containing monomers, in an aspect acrylic acid are particularly beneficial,
(α2) in one aspect 0 wt. % to about 70 wt. %, in another aspect about 1 wt. % to about 60 wt. %, and in yet another aspect about 1 wt. % to about 40 wt. % of polymerized, ethylenically unsaturated monomers which are copolymerizable with (α1),
(α3) in one aspect about 0.001 wt. % to about 10 wt. %, in another aspect about 5 0.01 wt. % to about 7 wt. % and in yet another aspect about 0.005 wt. % to about 5 wt. % of one or more crosslinkers,
(α4) in one aspect 0 wt. % to about 30 wt. %, in another aspect about 1 wt. % to about 20 wt. % and in yet another aspect about 5 wt. % to about 10 wt. % of water-soluble polymers, as well as
(α5) in one aspect 0 wt. % to about 20 wt. %, in another aspect about 0.01 wt. % to about 7 wt. %, and in yet another aspect about 0.05 to about 5 wt. % of one or more auxiliaries,
   whereby the sum of the weight quantities (α1) to (α5) amounts to about 100 wt. %.

The monoethylenically unsaturated, acid group-containing monomers (α1) can be, partially or fully, in an aspect partially neutralized. The monoethylenically unsaturated, acid group-containing monomers can be neutralized in one aspect to at least about 25 mol. %, in another aspect to at least about 50 mol. %, and in yet another aspect to at least about 50 mol. % to about 90 mol. %. The neutralization of the monomers (α1) can occur before and also after the polymerization. Further, the neutralization can occur with alkali metal hydroxides, alkaline earth metal hydroxides, ammonia as well as carbonates and bicarbonates. In addition, every further base is conceivable which forms a water-soluble salt with the acid. A mixed neutralization with different bases is also conceivable. Neutralization with ammonia or with alkali metal hydroxides is one aspect, and another aspect is with sodium hydroxide or with ammonia.

The monoethylenically unsaturated, acidic group-containing monomers (α1) can be acrylic acid, methacrylic acid, ethacrylic acid, α-chloroacrylic acid, a-cyanoacrylic acid, β-methylacrylic acid (crotonic acid), α-phenylacrylic acid, β-acryloxypropionic acid, sorbinic acid, α-chlorosorbinic acid, 2′-methylisocrotonic acid, cinnamic acid, p-chlorocinnamic acid, β-stearic acid, itaconic acid, citraconic acid, mesaconic acid, glutaconic acid, aconitic acid, maleic acid, fumaric acid, tricarboxythylene and maleic acid anhydride, wherein acrylic acid and methacrylic acid are an aspect, and acrylic acid is another aspect.

Besides these carboxylate group-containing monomers, further monoethylenically unsaturated acidic group-containing monomers (α1) are ethylenically unsaturated sulfonic acid monomers or ethylenically unsaturated phosphonic acid monomers.

The ethylenically unsaturated sulfonic acid monomers can be allylsulfonic acid or aliphatic or aromatic vinylsulfonic acids or acrylic or methacrylic acids. The aliphatic or aromatic vinylsulfonic acids can be vinylsulfonic acid, 4-vinylbenzylsulfonic acid, vinyltoluenesulfonic acid and styrenesulfonic acid. The acrylic or methacrylic sulfonic acids can be sulfoethyl(meth)acrylate, sulfopropyl(meth)acrylate and 2-hydroxy-3-methacryloxypropylsulfonic acid. As (meth)acrylamidoalkylsulfonic acid, 2-acrylamido-2-methylpropanesulfonic acid is an aspect.

Additionally, examples of ethylenically unsaturated phosphonic acid monomers include vinylphosphonic acid, allylphosphonic acid, vinylbenzylphosphonic acid, (meth)acrylamidoalkylphosphonic acids, acrylamidoalkyldiphosphonic acids, phosphonomethylated vinylamines and (meth)acrylphosphonic acid derivatives.

In one aspect of the invention, the water-absorbing polymer comprises at least about 50 wt. %, in another aspect at least about 70 wt. %, and in yet another aspect at least about 90 wt. % carboxylate group-containing monomers. In an embodiment of the invention, the water-absorbing polymer comprises in one aspect at least about 50 wt. %, in another aspect at least about 70 wt. % acrylic acid, which is neutralized in one aspect to at least about 20 mol. %, in another aspect to at least about 50 mol. %, and in yet another aspect within the range of about 65 mol. % to about 85 mol. %, in one aspect with sodium hydroxide.

Ethylenically unsaturated monomers (α1) containing a protonated nitrogen include dialkylaminoalkyl(meth)acrylates in the protonated form, for example dimethylaminoethyl(meth)acrylate hydrochloride or dimethylaminoethyl(meth)acrylate hydrosulfate, as well as dialkylaminoalkyl(meth)acrylamides in the protonated form, for example dimethylaminoethyl(meth)acrylamide hydrochloride, dimethylaminopropyl(meth)acrylamide hydrochloride, dimethylaminopropyl(meth)acrylamide hydrosulfate, or dimethylaminoethyl(meth)acrylamide hydrosulfate.

Ethylenically unsaturated monomers (α1) containing a quatemated nitrogen include dialkylammoniumalkyl(meth)acrylates in quatemated form, for example trimethylammoniumethyl(meth)acrylate methosulfate or dimethylethylammoniumethyl(meth)acrylate ethosulfate as well as (meth)acrylamidoalkyldialkylamine in quatemated form, for example (meth)acrylamidopropyltrimethylammonium chloride, trimethylammoniumethyl(meth)acrylate chloride, or (meth)acrylamidopropyltrimethylammonium sulfate.

Monoethylenically unsaturated monomers (α2) which can be co-polymerized with (α1) include acrylamides and (meth)acrylamides.

Possible (meth)acrylamides besides acrylamide and methacrylamide include alkyl-substituted (meth)acrylamides or aminoalkyl-substituted derivatives of (meth)acrylamide such as N-methylol(meth)acrylamide, N,N-dimethylamino(meth)acrylamide, dimethyl(meth)acrylamide or diethyl(meth)acrylamide. Possible vinylamides are for example N-vinylamides, N-vinylformamides, N-vinylacetamides, N-vinyl-N-methylacetamides, N-vinyl-N-methylformamides, and/or vinylpyrrolidone. Among these monomers acrylamide is one aspect.

Further, monoethylenically unsaturated monomers (α2) which are copolymerizable with (α1) include water-dispersible monomers. Water-dispersible monomers include acrylic acid esters and methacrylic acid esters, such as methyl(meth)acrylate, ethyl(meth)acrylate, propyl(meth)acrylate or butyl(meth)acrylate, as well as vinylacetate, styrene, and isobutylene.

Cross-linkers (α3) according to an embodiment of the invention include compounds which have at least two ethylenically unsaturated groups in one molecule (cross-linker class I), compounds which have at least two functional groups which can react with functional groups of the monomers (α1) or (α2) in a condensation reaction (=condensation cross-linkers), in an addition reaction or a ring-opening reaction (cross-linker class II), compounds which have at least one ethylenically unsaturated group and at least one functional group which can react with functional groups of the monomers (α1) or (α2) in a condensation reaction, an addition reaction or a ring-opening reaction (cross-linker class III), or polyvalent metal cations (cross-linker class IV). Thus with the compounds of cross-linker class I a cross-linking of the polymer can be achieved by radical polymerization of the ethylenically unsaturated groups of the cross-linker molecules with the monoethylenically unsaturated monomers (α1) or (α2), while with the compounds of cross-linker class II and the polyvalent metal cations of cross-linker class IV, a cross-linking of the polymer, can be achieved respectively via condensation reaction of the functional groups (cross-linker class II) or via electrostatic interaction of the polyvalent metal cation (cross-linker class IV) with the functional groups of the monomer (α1 ) or (α2). With compounds of cross-linker class III a cross-linking of the polymers can be achieved correspondingly by radical polymerization of the ethylenically unsaturated groups, as well as by condensation reaction between the functional groups of the cross-linkers and the functional groups of the monomers (α1) or (α2).

Examples of compounds of cross-linker class I include poly(meth)acrylic acid esters, which have been obtained for example by conversion of a polyol, such as for example ethylene glycol, propylene glycol, trimethylolpropane, 1,6-hexanediol, glycerine, pentaerythritol, polyethyleneglycol or polypropyleneglycol, of an aminoalcohol, a polyalkylenepolyamine, such as for example diethylenetriamine or triethylenetetraamine, or of an alkoxidised polyol with acrylic acid or methacrylic acid. Further compounds of cross-linker class I include polyvinyl compounds, poly(meth)allyl compounds, (meth)acrylic acid esters of a monovinyl compound or (meth)acrylic acid esters of a mono(meth)allyl compound, in an aspect of the mono(meth)allyl compounds of a polyol or of an aminoalcohol. In this context, reference is made to DE 195 43 366 and DE 195 43 368.

Examples of compounds of cross-linker class I include those named alkenyldi(meth)acrylates, for example ethyleneglycoldi(meth)acrylate, 1,3-propyleneglycoldi(meth)acrylate, 1,4-butyleneglycoldi(meth)acrylate, 1,3-butyleneglycoldi(meth)acrylate, 1,6-hexanedioldi(meth)acrylate, 1,10-decanedioldi(meth)acrylate, 1,12-dodecanedioldi(meth)acrylate, 1,18-octadecanedioldi(meth)acrylate, cyclopentanedioldi(meth)acrylate, neopentylglycoldi(meth)acrylate, methylenedi(meth)acrylate or pentaerythritoldi(meth)acrylate, alkenyldi(meth)acrylamides, for example N-methyldi(meth)acrylamide, N,N′-3-methylbutylidenebis(meth)acrylamide, N,N′-(1,2-dihydroxyethylene)bis(meth)acrylamide, N,N′-hexamethylenebis(meth)acrylamide or N,N′-methylenebis(meth)acrylamide, polyalkoxydi(meth)acrylates, for example diethyleneglycoldi(meth)acrylate, triethyleneglycoldi(meth)acrylate, tetraethyleneglycoldi(meth)acrylate, dipropyleneglycoldi(meth)acrylate, tripropyleneglycoldi(meth)acrylate or tetrapropyleneglycoldi(meth)acrylate, bisphenol-A-di(meth)acrylate, ethoxylated bisphenol-A-di(meth)acrylate, ethoxylated bisphenol-A-di(meth)acrylate, benzylidenedi(meth)acrylate, 1,3-di(meth)acryloyloxypropanol-2, hydroquinonedi(meth)acrylate, di(meth)acrylate esters of trimethylolpropane which is in one aspect alkoxylated with 1 to 30 mol alkylene oxide per hydroxyl group and in another aspect ethoxylated, thioethyleneglycoldi(meth)acrylate, thiopropyleneglycoldi(meth)acrylate, thiopolyethyleneglycoldi(meth)acrylate, thiopolypropyleneglycoldi(meth)acrylate, divinyl ethers, for example 1,4-butanedioldivinyl ether, divinyl esters, for example divinyl adipate, alkanedienes, for example butadiene or 1,6-hexadiene, divinylbenzene, di(meth)allyl compounds, for example di(meth)allyl phthalate or di(meth)allyl succinate, homo- and co-polymers of di(meth)allyldimethylammonium chloride and homo- and co-polymers of diethyl(meth)allylaminomethyl(meth)acrylateammonium chloride, vinyl(meth)acrylic compounds, for example vinyl(meth)acrylate, (meth)allyl(meth)acrylic compounds, for example (meth)allyl(meth)acrylate, (meth)allyl(meth)acrylate ethoxylated with 1 mol. to 30 mol. ethylene oxide per hydroxyl group, di(meth)allyl esters of polycarboxylic acids, for example di(meth)allyl maleate, di(meth)allyl fumarate, di(meth)allyl succinate or di(meth)allyl terephthalate, compounds with 3 or more ethylenically unsaturated, radically polymerizable groups such as for example glycerine tri(meth)acrylate, (meth)acrylate esters of glycerine ethoxylated in one aspect with about 1 mol. to about 30 mol. ethylene oxide per hydroxyl group, trimethylolpropanetri(meth)acrylate, tri(meth)acrylate esters of trimethylolpropane which is alkoxylated in one aspect with about 1 mol. to about 30 mol. alkylene oxide per hydroxide group and beneficially ethoxylated, trimethacrylamide, (meth)allylidenedi(meth)acrylate, 3-allyloxy- 1,2-propanedioldi(meth)acrylate, tri(meth)allylcyanurate, tri(meth)allylisocyanurate, pentaerythritoltetra(meth)acrylate, pentaerythritoltri(meth)acrylate, (meth)acrylic acid esters of pentaerythritol which is ethoxylated in one aspect with about 1 mol. to about 30 mol. ethylene oxide per hydroxyl group, tris(2-hydroxyethyl)isocyanuratetri(meth)acrylate, trivinyltrimellitate, tri(meth)allylamine, di(meth)allylalkylamines, for example di(meth)allylmethylamine, tri(meth)allylphosphate, tetra(meth)allylethylenediamine, poly(meth)allyl ester, tetra(meth)allyloxyethane or tetra(meth)allylammonium halides. Accordingly, in one aspect, the crosslinker class I includes vinylisocyanate, trivinyltrimellitate or tri(meth)allylisocyanurate, whereby trivinyltrimellitate is an aspect.

Examples of compounds of cross-linker class II include compounds which have at least two functional groups which can react in a condensation reaction (=condensation cross-linkers), in an addition reaction or in a ring opening reaction with the functional groups of the monomers (α1) or (α2), in an aspect with acidic groups of the monomers (α1) . These functional groups of the compounds of cross-linker class II are alcohol, amine, aldehyde, glycidic, isocyanate, carbonate, or epichloro functions.

Examples of compounds of cross-linker class II include polyols, for example ethylene glycol, polyethylene glycols such as diethylene glycol, triethylene glycol and tetraethylene glycol, propylene glycol, polypropylene glycols such as dipropylene glycol, tripropylene glycol or tetrapropylene glycol, 1,3-butanediol, 1,4-butanediol, 1,5-pentanediol, 2,4-pentanediol, 1,6-hexanediol, 2,5-hexanediol, glycerine, polyglycerine, trimethylolpropane, polyoxypropylene, oxyethylene-oxypropylene block copolymers, sorbitan-fatty acid esters, polyoxyethylene sorbitan-fatty acid esters, pentaerythritol, polyvinylalcohol and sorbitol, aminoalcohols, for example ethanolamine, diethanolamine, triethanolamine or propanolamine, polyamine compounds, for example ethylenediamine, diethylenetriamine, triethylenetetraamine, tetraethylenepentaamine or pentaethylenehexaamine, polyglycidyl ether compounds such as ethyleneglycoldiglycidyl ether, polyethyleneglycoldiglycidyl ether, glycerinediglycidyl ether, glycerinepolyglycidyl ether, pentaerithritolpolyglycidyl ether, propyleneglycoldiglycidyl ether, polypropyleneglycoldiglycidyl ether, neopentylglycoldiglycidyl ether, hexanediolglycidyl ether, trimethylolpropanepolyglycidyl ether, sorbitolpolyglycidyl ether, phthalic acid diglycidyl ester, adipinic acid diglycidyl ether, 1,4-phenylenebis(2-oxazoline), glycidol, polyisocyanates, in an aspect diisocyanates such as 2,4-toluenediioscyanate and hexamethylenediisocyanate, polyaziridine compounds such as 2,2-bishydroxymethylbutanol-tris[3-(1 -aziridinyl)propionate], 1,6-hexamethylenediethylene urea and diphenylmethane-bis-4,4′-N,N′-diethylene urea, halogen epoxides for example epichloro- and epibromohydrin and α-methylepichlorohydrin, alkylene carbonates such as 1,3-dioxolane-2-one (ethylene carbonate), 4-methyl-1,3-dioxolane-2-one (propylene carbonate), 4,5-dimethyl-1,3-dioxolane-2-one, 4,4-dimethyl-1,3-dioxolane-2-one, 4-ethyl-1,3-dioxolane-2-one, 4-hydroxymethyl-1,3-dioxolane-2-one, 1,3-dioxane-2-one, 4-methyl-1,3-dioxane-2-one, 4,6-dimethyl-1,3-dioxane-2-one, 1,3-dioxolane-2-one, poly-1,3-dioxolane-2-on, polyquatemary amines such as condensation products from dimethylamines, and epichlorohydrin. Further compounds of the cross-linker class II include in addition polyoxazolines such as 1,2-ethylenebisoxazoline, cross-linkers with silane groups such as γ-glycidoxypropyltrimethoxysilane and γ-aminopropyltrimethoxysilane, oxazolidinones such as 2-oxazolidinone, bis- and poly-2-oxazolidinone, and diglycolsilicates.

Compounds of class III include hydroxyl or amino group-containing esters of (meth)acrylic acid, such as for example 2-hydroxyethyl(meth)acrylate, as well as hydroxyl or amino group-containing (meth)acrylamides, or mono(meth)allylic compounds of diols.

The polyvalent metal cations of cross-linker class IV are derived from singly or multiply charged cations, the singly charged in particular from alkali metals such as potassium, sodium, and/or lithium, wherein lithium is an aspect. Doubly charged cations are derived from zinc, beryllium, alkaline earth metals such as magnesium, calcium, and/or strontium, wherein magnesium is an aspect. Further cations applicable according to an embodiment of the invention, with higher charge, are cations from aluminium, iron, chromium, manganese, titanium, zirconium, and other transition metals, as well as double salts of such cations or mixtures of the named salts. The use of aluminium salts and alums and various hydrates thereof such as e.g. AlCl₃ .6 H₂O, NaAl(SO₄)₂.12 H₂O, KAl(SO₄)₂.12 H₂O or Al₂(SO₄)₃.14-18 H₂O is an aspect.

The use of Al₂(SO₄)₃ and its hydrates as cross-linkers of the cross-linker class IV is an aspect.

Water-absorbing polymers include those which are cross-linked respectively by cross-linkers of the following cross-linker classes or by cross-linkers of the following combinations of cross-linker classes: I, II, III, IV, I II, I III, I IV, I II III, I II IV, I III IV, II III IV, II IV, or III IV. The above combinations of cross-linker classes represent respective aspects of an embodiment of cross-linkers of a water-absorbing polymer particle.

A further embodiment of the invention includes water-absorbing polymers cross-linked by any of the above named cross-linkers of cross-linker class I. Among these, water soluble cross-linkers are an aspect. In this context, N,N′-methylenebisacrylamide, polyethylene glycol di(meth)acrylates, triallylmethylammonium chloride, tetraallylammonium chloride as well as allylnonaethylene glycol acrylate made with about 9 mol ethylene oxide per mol acrylic acid are another aspect.

As water soluble polymers (α4), water soluble polymers such as partially or fully saponified polyvinyl alcohol, polyvinylpyrrolidone, starches or starch derivatives, polyglycols, or polyacrylic acids can comprise, in an aspect, can be polymerized into, the water-absorbing polymer according to an embodiment of the invention. The molecular weight of these polymers is not critical as long as they are water soluble. Water soluble polymers include starches or starch derivatives or polyvinyl alcohol. Also, water soluble polymers, in an aspect synthetic polymers such as polyvinyl alcohol, can serve as a graft basis for the monomers to be polymerized.

As additives (α5), suspension agents, odor binders, surface-active agents, or antioxidants can be used.

The water-absorbing polymer can be produced from the above-named monomers and cross-linkers by various polymerization means. For example, in this context can be named bulk polymerization which occurs in an aspect in kneading reactors such as extruders or by belt polymerization, solution polymerization, spray polymerization, inverse emulsion polymerization, and inverse suspension polymerization. Solution polymerization is carried out in an aspect in water as a solvent. The solution polymerization can occur continuously or discontinuously, as can the other above-mentioned polymerization types. The solution polymerization in another aspect can occur as continuously running belt polymerization. From the art, a broad spectrum of variational possibilities can be learned with respect to reaction proportions such as temperatures, type, and quantity of the initiators as well as of the reaction solution. Typical processes are described in the following patent specifications: U.S. Pat. No. 4,286,082, DE 27 06 135, U.S. Pat. No. 4,076,663, DE 35 03 458, DE 40 20 780, DE 42 44 548, DE 43 23 001, DE 43 33 056, and/or DE 44 18 818. In the case of the above-mentioned polymerization types, in an aspect the wound-treatment substance can be already introduced into the polymerization as a mixture with the solvent or monomer or respective monomers used in the corresponding polymerization process in the variants already described above.

Another possibility for producing the water-absorbing polymers is to first produce non-crosslinked, in particular linear pre-polymers, by radical means from the above-mentioned monoethylenically unsaturated monomers (α1) or (α2) and then to convert these with reagents acting as a crosslinker (α3), in an aspect with those of classes II and IV. This variant then can be used if the water-absorbing polymer should be first processed in form-giving processes, for example into fibers, films, or other flat structures such as fabrics, woven fabrics, webs or non-woven materials, and cross-linked in this form. In the context of these production processes, respectively an active substance or a care substance can on the one hand be incorporated in the production of the pre-polymer. On the other hand, the respective active substance or the care substance can be incorporated in the crosslinking of the pre-polymer.

In the production of the water-absorbing polymers, solvents known to the skilled person for producing such polymers, in an aspect water, the common temperature ranges, in one aspect with a temperature of about 1 to about 100° C., in another aspect from about 3 to about 600° C., and pressures in the normal pressure range are used.

The water-absorbing polymer obtainable by above processes can, for example, if a more solid gel is desired, undergo a further crosslinking reaction in which a part of the acid groups of this polymer, in an aspect in the area of the surface, are crosslinked by at least bifunctional compounds. This reaction is generally characterized as “post-crosslinking.” For details of the post-crosslinking, in addition to the following, reference is made to DE 40 20 780 C1. The post-crosslinking is for example available, if the active substance-doped water-absorbing polymer particles according to embodiments of the invention or the composition according to embodiments of the invention are subjected to increased pressure during the wound healing, as can for example be the case with pressure dressings.

Embodiments of the polymers include those which are post-crosslinked by means of crosslinkers of the following crosslinker classes or respectively by crosslinkers of the following combinations of crosslinker classes: II, IV and II IV. In one aspect, post-crosslinkers include the compounds of crosslinker classes II and IV mentioned in context of the crosslinkers.

Among these compounds, in another aspect as post-crosslinker include diethylene glycol, triethylene glycol, polyethylene glycol, glycerine, polyglycerine, propylene glycol, diethanolamine, triethanolamine, polyoxypropylene, oxyethylene-oxypropylene block copolymers, sorbitan fatty acid esters, polyoxyethylene sorbitan fatty acid esters, trimethylolpropane, penthaerythritol, polyvinyl alcohol, sorbitol, 1,3-dioxolane-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxane-2-one, 4-methyl-1,3-dioxane-2-one, 4,6-dimethyl- 1,3-dioxane-2-one, 1,3-dioxolane-2-one, and/or poly-1,3-dioxolane-2-one.

In yet another aspect, ethylene carbonate is used as a post-crosslinker.

Further, embodiments of the invention relating to polymers are those which are post-crosslinked by any of the above-mentioned crosslinkers in the crosslinker classes II or IV.

These compounds are used in one aspect in a quantity in the range from about 0.01 wt. % to about 30 wt. %, in another aspect from about 0.1 wt. % to about 20 wt. %, and in yet another aspect from about 0.5 wt. % to about 10 wt. %, based on the as yet untreated polymer. Organic solvents can be added to the mixture in a quantity in one aspect from 0 wt. % to about 60 wt. %, in another aspect from about 0.1 wt. % to about 40 wt. %, and in yet another aspect from about 0.2 wt. % to about 50 wt. %, based on the as yet untreated polymer. Organic solvents may include lower alcohols such as methanol, ethanol, n-propanol, isopropanol, n-butanol, isobutanol, sec-butanol and t-butanol; ketones such as acetone, methyl ethyl ketone and methylisobutylketone; ethers such as dioxane, tetrahydrofuran and diethyl ether; amides such as N,N-dimethylformamide and N,N-diethylformamide, or sulfoxides such as dimethylsulfoxide.

In addition, a water-absorbing composition according to an embodiment of the invention, the polycondensate matrix comprises a polyurethane that, in one aspect comprises at least about 10 wt. %, in another aspect to at least about 50 wt. %, and in yet another aspect to at least about 80 wt. %, respectively based on the polycondensate matrix. In this context, the part which is different from polyurethane can be another polymer different from the composition components, with which the polyurethane is mixed for example by co-extrusion. The other polymer is in an aspect further a water-insoluble, non-water-swellable thermoplastic polymer.

All polyurethanes suitable according to the skilled person are considered as polyurethanes according to embodiments of the invention. Suitable polyurethanes are obtainable at least from the polyurethane components:
(γ1) One or more polyisocyanates with at least two isocyanate groups, such as methylene diphenyl diisocyanate (MDI), tolueylene diisocyanate (TDI) or isophorone diisocyanate (IPDI), or a mixture of at least two thereof;
(γ2) One or more polyhydroxyl compounds, such as with an average OH number in the range from about 20 to about 112;
(γ3) optionally one or more catalysts or accelerators, respectively for the reaction between isocyanate and hydroxyl groups; as well as
(γ4) optionally filling materials and additives known in polyurethane chemistry.

Further details concerning the above-mentioned (γ1) to (γ4) are given in DE 42 33 289 A1 as well as DE 196 18 825 A1. This is particularly the case for the details regarding polyurethane gels and their production in these documents.

The water-absorbing composition according to embodiments of the invention and the active substance-doped water-absorbing polymer particles according to an embodiment of the invention have in one aspect at least one, and in another aspect all of the following properties:
C1) A residual monomer content of a monomer used for forming the water-absorbing polymer in one aspect of less than about 500 ppm, in another aspect less than about 250 ppm, and in yet another aspect less than about 146 ppm, respectively based on the water-absorbing polymer used in the water-absorbing composition or respectively in the active substance-doped water-absorbing polymer particles, determined according to ERT 410.1-99;
C2) A soluble polymer fraction after about 16 hours extraction, based on the monomers used for forming the water-absorbing polymers in one aspect of less than about 30 wt. %, in another aspect less than about 20 wt. %, in yet another aspect less than about 15 wt. %, and in even yet another aspect less than about 12 wt. %, respectively based on the water-absorbing polymer used in the water-absorbing composition or respectively in the active substance-doped water-absorbing polymer particles, determined according to ERT 470.1-99;
C3) an active substance or respective care substance availability in one aspect of at least about 40 wt. %, in another aspect at least about 70 wt. %, in yet another aspect at least about 85 wt. %, and in even yet another aspect at least about 95 wt. % of a respective active substance or care substance comprised in the water-absorbing composition or in the active substance-doped water-absorbing polymer particles, determined according to the herein described extraction test;
C4) a liquid absorption in one aspect of at least about 1, in another aspect at least about 4, and in yet another aspect at least about 9 g/100cm²;
C5) a water vapor permeability in one aspect of at least about 100, in another aspect at least about 200, and in yet another aspect at least about 249 g/(m²×24 h); or
C6) an oxygen permeability in one aspect of at least about 100, in another aspect at least about 1000, and in yet another aspect at least about 1999 cm³/(m²×24 h).

Each of the feature combinations generated from the features C1 to C6 represents embodiments of the invention, whereby the following feature combinations represent respective aspects of embodiments: C2C3C4C5C6, C1C3C4C5C6, C1C2C3C4C6, C1C2C4C5C6, C1C4C5C6, C2C4C5C6, C3C4C5C6, C1C4C5, C1C5C6, C3C5C6, C4C5C6, C3C4, C3C5, C3C6, C1C2, C2C3, or C1C3.

It is furthermore an aspect that in the water-absorbing composition according to an embodiment of the invention, the polycondensate matrix is present as a foam. The foam is obtained by means of blowing agents known for this purpose to the skilled person in the course of the formation of the polyurethane. In this context, reference is made to the corresponding details in DE 42 33 289 A1 regarding foaming means and their use.

Further embodiments of the invention relate to a composite, comprising a water-absorbing composition according to an embodiment of the invention or the active substance-doped water-absorbing polymer particles.

A design of the composite has in one aspect at least one, and in another aspect all of the following properties:
V1) a viscous elasticity in one aspect in the range from about 0.1 to about 10, in another aspect in the range from about 0. 1 5 to about 7, and in yet another aspect in the range from about 2 to about 5 tan δ (ω=0.3 rad/s);
V2) a liquid absorption in one aspect of at least about 5, in another aspect of at least about 8, and in yet another aspect in the range of about 10 to about 1000, as well as in even yet another aspect in the range from about 10.5 to about 20 g/100cm²;
V3) a water vapor permeability in one aspect of at least about 100 g/(m²×24 h) and in another aspect at least about 200 g/(m²×24 h);
V4) an O₂ permeability in one aspect of at least about 100, in another aspect of at least about 1000, and in yet another aspect in the range from about 1100 to about 10000, as well as in even yet another aspect in the range from about 1500 to about 3000 cm³/(m²×24 h).

Each of the feature combinations originating from the features V1 to V4 represent embodiments of the invention relating to a composite, whereby the following feature combinations represent respective aspects of embodiments: V1V2, V1V3, V1V4, V1V2V3, V1V2V4, V1V3V4, V2V3V4, or V1V2V3V4.

In an embodiment of the invention, the composite comprises, besides the composition according to an embodiment of the invention or the polymer particles according to an embodiment of the invention, also a film, which can be directly adjacent to the composition or the polymer particles. The composition or the polymer particles in the form of a thin layer fill in one aspect at least about 40 %, in another aspect at least about 70 %, and in yet another aspect at least about 90 % of the surface of a side of the film. Further, the film and respectively the composition or the polymer particles can be bound firmly with each other. This can occur by the adhesive effect of the matrix on the composition. Further, the film can have a water vapor permeability in one aspect in the range from about 100 to about 2000 g/(m²×24 h), in another aspect in the range from about 300 to about 1500 g/(m²×24 h), and in yet another aspect in the range from about 500 to about 10 00 g/(m²×24 h). As film are considered all materials common to the skilled person and known to be suitable. In an aspect, a film comprises a plastic film, and in another aspect a film principally comprises polyethylene or polypropylene or mixtures thereof.

If the composite is an absorbent core—also named absorbent layer (“Sauglage”)—or generally in a core, the active substance-doped water-absorbing polymer particles according to embodiments of the invention, the composition according to embodiments of the invention or also the composite according to embodiments of the invention is incorporated into a substrate. The active substance-doped water-absorbing polymer particle according to embodiments of the invention can be present therein in one aspect to at least about 5 wt. %, in another aspect in the range from about 6 to about 50 wt. %, as well as in yet another aspect in the range from about 7 to about 15 wt. %, respectively based on the core. In a further design of an embodiment of the present invention, in one aspect at least about 10 wt. %, in another aspect at least about 30 wt. %, and in yet another aspect at least about 60 wt. %, as well as in even yet another aspect at least about 80 wt. %, respectively based on the total weight of the water-absorbing polymer in the core, of water-absorbing polymer particles which are not doped with active substance are mixed with the active substance-doped water-absorbing polymer particles according to an embodiment of the invention. In addition, the core can comprise water-absorbing polymer, in an aspect as particles, in one aspect in the range of about 40 to about 95 wt. %, in another aspect in the range of about 50 to about 90 wt. %, and in yet another aspect in the range from about 75 to about 85 wt. %, respectively based on the core.

This substrate is in an aspect fiber materials. Fiber materials that can be used in embodiments of the present invention comprise naturally occurring fibers (modified or unmodified) as well as synthetic fibers. Examples of suitable unmodified and modified natural fibers comprise cotton, esparto grass, sugar cane, hemp, flax, silk, wool, cellulose, chemically modified cellulose, jute, rayon, ethyl cellulose, and cellulose acetate. Suitable synthetic fibers can be produced from polyvinylchloride, polyvinylfluoride, polytetrafluoroethylene, polyvinylidine chloride, polyacrylates such as Orion® polyvinylacetate, polyethylvinylacetate, insoluble or soluble polyvinylalcohol, polyolefines, such as polyethylene (for example PULPEX®) and polypropylenes, polyamides, such as Nylon, polyesters such as DACRON® or Kodel®, polyurethanes, polystyrenes, and the like. The fibers used can comprise only naturally occurring fibers, only synthetic fibers or any compatible combination of natural and synthetic fibers.

Besides the above-mentioned fiber materials, the core can also comprise thermoplastic materials. On melting, at least a part of this thermoplastic material moves around, typically caused by the capillary gradients between the fibers through to the intersections of the fibers. These intersections become binding positions for the thermoplastic material. When the element is cooled, the thermoplastic material solidifies at these intersections to form binding positions which hold together the matrix or the web of fibers in each of the respective layers.

The thermoplastic materials can be present in different forms, such as particles, fibers, or combinations of particles and fibers. These materials can be selected from a plurality of thermoplastic polymers from polyolefins, such as polyethylene (for example PULPEX®) and polypropylene, polyesters, copolyesters, polyvinylacetates, polyethylvinylacetates, polyvinylchlorides, polyvinylidenechlorides, polyacrylates, polyamides, copolyamides, polystyrenes, polyurethanes, and copolymers of the above materials, such as vinyl chloride/vinyl acetate and the like. For cores, substrates are predominantly considered materials comprising cellulose, in an aspect fibrous materials.

The core can be produced, on the one hand, by a so called Airlaid process or by a so-called Wetlaid process, wherein a core produced according to the Airlaid process can be one aspect. In the Wetlaid process, the fibers or particles of absorbent polymer structure, together with further substrate fibers and a liquid, are processed into a non-woven material. In the Airlaid process, the fibers or particles of absorbent polymer structure and the substrate fibers in the dry state are processed into a non-woven material. Further details concerning Airlaid processes are described in U.S. Pat. No. 5,916,670, as well as U.S. Pat. No. 5,866,242, and concerning Wetlaid processes in U.S. Pat. No. 5,300,192.

A further embodiment of the present invention is a hygiene article, in one aspect baby diapers, sanitary napkins, tampons, or adult diapers, and in another aspect baby diapers, comprising an active substance-doped water-absorbing polymer particle according to an embodiment of the invention or a composition according to an embodiment of the invention or a composite according to an embodiment of the invention, or at least two thereof. In general, a hygiene article used as a diaper comprises a water-impermeable lower layer, in an aspect a water-permeable, and in another aspect a hydrophobic, upper layer, and a layer comprising at least one active substance-doped water-absorbing polymer particle according to embodiments of the invention, or one composition according to embodiments of the invention, or a composite according to embodiments of the invention or at least two thereof, which is arranged between the lower layer and the upper layer. This layer can be a previously described core. The lower layer can comprise all materials known to the skilled person, whereby polyethylene or polypropylene are aspects. The upper layer can likewise comprise all materials known and suitable to the skilled person, whereby polyesters, polyolefins, viscose, and the like are aspects, which give a sufficiently porous layer to ensure a sufficient liquid permeability of the upper layer. In this context, reference is made to the disclosure in U.S. Pat. No. 5,061,295, U.S. Re. 26,151, U.S. Pat. No. 3,592,194, U.S. Pat. No. 3,489,148, and U.S. Pat. No. 3,860,003.

In addition, an embodiment of the present invention relates to the use of a water-absorbing composition according to an embodiment of the invention for release of an active substance, in one aspect a wound treatment substance or a care substance. The invention also relates to the use of an active substance-doped polymer particle according to an embodiment of the invention for release of a care substance, in one aspect a skin care substance. The invention further relates to the use of an above-described water-absorbing polymer according to an embodiment of the invention for release of an active substance, in one aspect a wound treatment substance or a care substance, from a polycondensate matrix. In this context, wound dressings and plasters are aspects.

An embodiment of the invention additionally relates to the use of a water-absorbing composition according to embodiments of the invention for producing a means for treating a wound of a higher vertebrate organism or for prevention of formation of a wound at or in a higher vertebrate organism. In one aspect, a higher vertebrate organism, according to an embodiment of the invention, is understood to be a fish, bird, mammal, or human, in another aspect a mammal or human, and in yet another aspect a human. The wounds can be cuts, abrasions, operation, or burn wounds, as well as in the case of hygiene articles, in particular of diapers, wounds arising from long periods of lying down and skin irritations.

In another embodiment of the invention, the invention further relates to a process for treating a wound of a higher vertebrate organism, for prevention of formation of a wound at or in a higher vertebrate organism, or for care of the skin of a higher vertebrate organism, whereby the wound or the skin is at least partially covered or brought into contact with a water-absorbing composition according to an embodiment of the invention, an active substance-doped polymer particle according to an embodiment of the invention, or a composite according to an embodiment of the invention.

Finally, an embodiment of the invention relates to the use of a water-absorbing composition according to embodiments of the invention, the use of an active substance-doped polymer particle according to embodiments of the invention, or the use of a composite according to embodiments of the invention in a hygiene article or in a wound treatment means.

The invention is more closely illustrated by means of the following examples which do not limit its scope of protection.

Test Methods

1. ERT

Unless otherwise stated in the following, ERT methods are used for determining the different properties relating to the water-absorbing polymer. “ERT” stands for EDANA Recommended Test, whereby “EDANA” stands for European Nonwoven And Diaper Association.

2. Extraction Test 0.5 g of an active substance-doped sample, in an aspect partially neutralized, lightly crosslinked polyacrylic acid with dexpanthenol as active substance or a polyurethane matrix comprising these, were weighed into a 125 ml wide-necked flask on an analytical balance. After addition of 100 ml of 0.9 % cooking salt solution (based on distilled water) and a drop of concentrated phosphoric acid, the mixture was stirred for one hour on the magnetic stirrer at 350 rpm. 2 ml of the solution was then removed and filtered through a 0.45 μm cellulose mix ester membrane filter in a sample vial. The filtrate was then analyzed by HPLC, whereby the sample used in the HPLC analysis had an acidic pH value in the range of 2.5 to 3.0.

The active substance content was determined using the HPLC analysis results by external calibration. To this end, the active substance to be determined was weighed using an analytical balance in a 100 ml graduated flask in an amount of at least 10 mg with an accuracy of 0.1 mg. The graduated flask was then filled with ultra pure water up to the mark. An array of dilutions corresponding to the concentration of the stock solution was then prepared on the analytical balance. By means of this array of dilutions, a calibration curve was generated by HPLC analysis. The quantity of active substance extracted over one hour was determined by comparison of the HPLC analysis results of the corresponding active substance with the calibration curve.

The chromatographic conditions were optimized depending on the active substance to be determined. In the case of dexpanthenol, a column of the type GromSil 300 ODS-5 5 μm (250×4 mm) was used. The eluent was produced by weighing 13.61 g KH₂PO₄ into a 3 1 beaker and dissolved after the addition of 2000 ml ultra pure water. The pH value was adjusted to 2.5 to 3.0 using concentrated phosphoric acid. In the case of dexpanthenol, a flow of 0.8 ml/min. was calibrated. The injection occurred via a loop of 20 μl.

3. Rheological Characterization

From the center of a plaster, a sample with a diameter of 8 mm was stamped and preconditioned for one hour at 23±2° C. and 50±5 % relative humidity. The sample was stuck in the center of an 8 mm plate turntable and measured using a shear stress-regulated rheometer with a thermoelectric (Peltier) (e.g. RS-75 from HAAKE) element for temperature control. To this end, the sample was pressed with a normal force of 1.3 N on the lower plate. After a conditioning of 5 minutes at 25±0.2° C., the viscous elastic properties (storage modulus and loss modulus) determined at a shear stress of 700 Pa in the frequency range ofω=0.3 to 30 rad/s. The tanδ is calculated from the quotients from loss and storage modulus.

4. Liquid Absorption

From the center of a plaster, a sample with a diameter of 15 mm was stamped and preconditioned for one hour at 23±2° C. and 50±5 % relative humidity. The samples are weighed and immersed completely for three hours in physiological 23±0.5° C. warm sodium chloride solution. The samples are reweighed and the liquid absorption calculated from the weight difference.

5. Water Vapor Permeability

The test is carried out according to ASTM E96 (water method), with the following differences: the opening of the test vessel is 804 mm². The material is preconditioned for 24 hours at 23±2° C. and 50±5 % relative humidity. The distance between the water level in the test vessel and the sample is 35±5 mm. The reweighing of the test vessel including this sample occurs after 24 hours, during which time it was stored in the climatic cabinet at 37±1.5° C. and 30±3 % relative humidity.

6. O₂ Permeability

The test was carried out according to ASTM D3985-81.

### EXAMPLES

1. Production of Dexpanthenol-Doped Water-Absorbing Polymer as Water-Absorbing Polymer Composition.

Variant A: Addition of Dexpanthenol to the Monomer Solution

0.45 g triallylamine as crosslinker was dissolved in 944.77 g of an aqueous solution of sodium acrylate with a degree of neutralization of 70 mol. % (monomer concentration of the preparation 36.4 %). 50 g of a 75% solution of dexpanthenol in water were then added. The monomer solution was flushed with nitrogen for 30 minutes in a plastic polymerization vessel, to remove therein-dissolved oxygen. At a temperature of 4° C. the polymerization was started by the successive addition of 0.25 g sodium peroxidisulfate in 10 g distilled water, 0.1 g 2,2′-azobis-2-amidinopropane dihydrochloride in 10 g distilled water, 0.2 g tert-butylhydroperoxide in 10 g distilled water, and 0.015 g ascorbic acid in 2 g distilled water. After the end temperature (100° C.) was reached, the gel was comminuted with a meat grinder (Fleischwolf) and dried in a circulating air oven (drying temperature and duration are given in the following table). The dried product was coarsely ground and finely ground, and the particles of the sizes from 150 to 850 μm sieved for further processing. For the incorporation of the absorbent polymer into a polyurethane matrix, the absorbent polymer powder was ground to a particle size of less than 300 μEm. The thus-obtained water-absorbing polymers have a residual moisture of 3 wt. %, based on the water-absorbing polymer.

Variant B: Addition of Dexpanthenol to the Polymer Gel

0.45 g triallylamine as crosslinker was dissolved in 993.08 grams of an aqueous solution of sodium acrylate with a degree of neutralization of 70 mol. % (monomer concentration of the preparation 36.4%). The monomer solution was flushed with nitrogen for 30 minutes in a plastic polymerization vessel, to remove dissolved oxygen. At a temperature of 4° C., the polymerization was started by successive addition of 0.25 g sodium peroxidisulfate in 10 g distilled water, 0.1 g 2,2′-azobis-2-amidinopropane dihydrochloride in 10 g distilled water, 0.2 g tert-butylhydroperoxide in 10 g distilled water and 0.015 g ascorbic acid in 2 g distilled water. After the end temperature (ca. 1 00° C.) was reached, the gel was comminuted with a meat grinder (Fleischwolf) and uniformly sprayed with 50 grams of a 75% solution of dexpanthenol in water and mixed. It was then dried in a circulating air oven (dry temperature and duration are given in the table), the dried product was coarsely ground and finely ground, and the particles with sizes of from 150 to 180 μm were sieved out for the further processing. For the incorporation of the superabsorber powder into a polyurethane matrix, the superabsorber was ground to a particle size of less than 300 μm. The thus-obtained water-absorbing polymers have a residual moisture of 3 wt. %, based on the water-absorbing polymer.

The residual moisture of the water-absorbing polymers detailed in the following table 1 was 3%.

TABLE 1

| | | | | | | Residual | |
|---|---|---|---|---|---|---|---|
| | | | Soluble | | | monomer | Extractable |
| | Dexpanthenol | Drying | fraction | CRC | AAP | content | dexpanthenol |
| | addition | ° C./min | 16 h [%] | [g/g] | [g/g] | [ppm] | [%]⁴ |
| Example 1 | 10%¹ | 120/210 | 11.9 | 34.0 | 7 | 145 | 80 |
| | (Variant A) | | | | | | |
| | 50 g² 75%³ | | | | | | |
| | solution | | | | | | |
| Example 2 | 10% | 120/210 | 12.1 | 35.2 | 7.2 | 210 | 91 |
| | (Variant B) | | | | | | |
| | 50 g 75% | | | | | | |
| | solution | | | | | | |
| Example 3 | 5% | 110/270 | 12.4 | 33.7 | 8 | 160 | 86 |
| | (Variant A) | | | | | | |
| | 25 g 75% | | | | | | |
| | solution | | | | | | |
| Example 4 | 5% | 120/210 | 12.3 | 33.7 | 7.8 | 155 | 98 |
| | (Variant B) | | | | | | |
| | 25 g 75% | | | | | | |
| | solution | | | | | | |

¹percent fraction dexpanthenol in superabsorber powder
²quantity of dexpanthenol solution used
³concentration of the dexpanthenol solution
⁴According to the extraction test, fraction extracted after one hour of the dexpanthenol originally used in the superabsorber powder

2. Production of a Wound Dressing with Dexpanthenol-Doped Superabsorber

2.1 Preparation of both Components:

TABLE 2

Wt. %

Component 1

| | |
|---|---|
| 82 | Polyetherpolyol * |
| 9 | Isocyanate prepolymer ** |
| 9 | Absorbent polymer according to |
| | example 1, doped with 10% |
| | dexpanthenol |

Component 2

| | |
|---|---|
| 90 | Polyetherpolyol * |
| 10 | Catalyst *** |

* pentaerythritol + propyleneoxide + ethylenoxide − mix polymer with ethylene oxide end block functionality: 4, OH-number: 35, average molecular weight: 6400 (calculated), viscosity (23° C.): 1000 mPas, ethylene oxide content: 20 wt. %
** NCO-terminated pre polymer from conversion at 80° C. of hexamethylenediisocyanate and polypropylene glycol (average molecular weight: 220) in a mol ratio 5:1 and then vacuum distillation at ca. 0.5 mbar (50 Pa) to a HDI monomer residual content less than 0.5 wt. % NCO content: 12.6 wt. %, viscosity (23° C.): 5000 mPas
*** solution of 1 Mol of the Bi(III) salt with 2,2-dimethyloctanoic acid in 3 mol 2,2-dimethylotanoic acid (bismuth content ca. 17 wt. %)

The quantities given in table 2 were weighed and mixed for 24 hours on a rolling fixture.

2.2 Production of Smears

98 wt. % of component 1 and 2 wt. % of component 2 were uniformly mixed for about 40 seconds by hand, placed on a release paper and smeared by means of a spreading bar with a gap adjustment of 1 mm between the release paper, and a plastic foil with a water vapor 5 permeability of 750 g/(m²×24 h) in order to then react for 5 minutes in a drying cupboard at 60° C. The thus-obtained smear has a surface weight of 850 g/m² and can be cut or stamped out into forms suitable for wound dressings or property measurements. The properties of the smear are given in table 3.

TABLE 3

| Property | Value ^{#} |
|---|---|
| Viscous elasticity | [tanδ (ω = 0.3 rad/s)] 0.225 |
| Liquid absorption | [g/100cm²] 12.5 |
| Water vapor permeability | [g/(m² × 24 h)] 350 |
| O₂ permeability | [cm³/(m² × 24 h)] 2050 |

^{#} average value from multiple determinations

1-21. (canceled)

22. Active substance-doped water-absorbing polymer particles comprising:
Φ1. an active substance in a quantity in the range from about 0.001 to about 30 wt. %, based on the active substance-doped water-absorbing polymer particles; and
Φ2. an absorber matrix in a quantity in the range from about 70 to about 99.999 wt. %, based on the active substance-doped water-absorbing polymer particles,
wherein the absorber matrix comprises a cross-linked polyacrylic acid to at least about 90 wt. %, based on the absorber matrix; and
wherein the cross-linked polyacrylic acid comprises, to at least about 90 wt. %, based on the cross-linked polyacrylic acid, an acrylic acid that is partially neutralized to at least about 30 mol. %.

23. Active substance-doped water-absorbing polymer particles according to claim 22, wherein the active substance comprises any one of a care substance, a wound-treating substance, or a care substance and a wound-treating substance.

24. Active substance-doped water-absorbing polymer particles according to claim 23, wherein the care substance comprises a skin care substance capable of any one of cleaning the skin, perfuming the skin, changing an appearance of the skin, protecting the skin, maintaining the skin in a good condition, or any combination of any of the preceding.

25. Active substance-doped water-absorbing polymer particles according to claim 22, wherein the active substance comprises a functional group including any one of a double bond, an OH group, an NH group, a COOH group, a salt of at least one of these groups, or any combination of any of the preceding.

26. Active substance-doped water-absorbing polymer particles according to claim 22, wherein the active substance comprises at least one wound-treating substance or a mixture of at least two wound-treating substances capable of disinfecting a wound area by any one of promoting homeostasis of a wound environment, stimulating cell growth in the wound area, stimulating a secretion of one or more proteins in the wound area, stimulating a secretion of proteoglucanes in the wound area, stimulating a secretion of messenger substances by the skin cells in the wound area, or any combination of any of the preceding.

27. Active substance-doped water-absorbing polymer particles according to claim 23, wherein the active substance comprises any one of an allantonin, a recutita, an arnica, a biotin, a coenzyme Q 10, a dexpanthenol, a honey or honey extract, an amino acid, a niacinamide, a vitamin C or its esters, a vitamin E or its esters, or any combination of any of the preceding.

28. Active substance-doped water-absorbing polymer particles according to claim 22, wherein the active substance comprises a substance substantially homogeneously distributed over the absorber matrix.

29. Active substance-doped water-absorbing polymer particles according to claim 22, wherein the active substance-doped water-absorbing polymer particles include a residual monomer content of the monomer on which the water-absorbing polymer particles are based of under about 500 ppm.

30. Active substance-doped water-absorbing polymer particles according to claim 22, wherein an active substance availability comprise at least about 40 wt. % according to the Extraction Test described herein.

31. A water-absorbing composition comprising:
Γ1. a polycondensate matrix based on at least one polycondensate monomer with at least one polycondensate group; and
Γ2. a particulate water-absorbing polymer comprising an active substance including at least one functional group that can react with at least one polycondensate group to form a covalent link; or a particulate water-absorbing polymer comprising:
Φ1. an active substance in a quantity in the range from about 0.001 to about 30 wt. %, based on the active substance-doped water-absorbing polymer particles; and
Φ2. an absorber matrix in a quantity in the range from about 70 to about 99.999 wt. %, based on the active substance-doped water-absorbing polymer particles,
wherein the absorber matrix comprises a cross-linked polyacrylic acid to at least about 90 wt. %, based on the absorber matrix; and
wherein the cross-linked polyacrylic acid comprises, to at least about 90 wt. %, based on the cross-linked polyacrylic acid, an acrylic acid that is partially neutralized to at least about 30 mol. %,
wherein the particulate water-absorbing polymer is at least partially surrounded by the polycondensate matrix;
wherein at least the particulate water-absorbing polymer comprises the active substance; and
wherein the water-absorbing composition has an active substance availability of at least about 10 wt. % according to the Extraction Test described herein.

32. A water-absorbing composition according to claim 31, wherein the active substance comprises any one of a care substance, a wound-treating substance, a salt of a care substance, a salt of a wound-treating substance, or any combination of any of the preceding.

33. A water-absorbing composition according to claim 31, wherein the water-absorbing polymer has at least one of the following properties:
A1) a particle size distribution, whereby at least about 80 wt. % of the particles have a particle size in a range from about 20 μm to about 900 μm according to ERT 420.1-99;
A2) a Centrifuge Retention Capacity (CRC) of at least about 10 g/g, preferably at least about 20 g/g according to ERT 441.1-99;
A3) an Absorption Against Pressure (AAP) at about 0.7 psi of at least about 4 g/g according to ERT 442.1-99;
A4) a water soluble polymer content after about 16 hours extraction of less than about 25 wt. %, based on the total weight of the water-absorbing polymer, according to ERT 470.1-99; or
A5) a residual moisture of at most about 15 wt. %, based on the total weight of the water-absorbing polymer, according to ERT 430.1-99.

34. A water-absorbing composition according to claim 31, wherein the water-absorbing polymer comprises:
(α1) about 0.1 to about 99.999 wt. % polymerized, ethylenically unsaturated, acidic group-containing monomers or salts thereof or polymerized, ethylenically unsaturated monomers comprising a protonated or quaternated nitrogen, or mixtures thereof,
(α2) 0 to about 70 wt. % polymerized, ethylenically unsaturated monomers copolymerizable with (α1),
(α3) about 0.001 to about 10 wt. % of one or more crosslinkers,
(α4) 0 to about 30 wt. % water soluble polymers, and
(α5) 0 to about 20 wt. % of one or more auxiliaries,
wherein the sum of the weight quantities (α1) to (α5) amounts substantially to about 100 wt. %.

35. A water-absorbing composition according to claim 31, wherein the polycondensate matrix comprises at least about 10 wt. %, based on the polycondensate matrix, a polyurethane.

36. A water-absorbing composition according to claim 31, wherein the polycondensate matrix comprises a foam.

37. A composite comprising a water-absorbing composition according to claim 31.

38. A composite according to claim 37, wherein the composite comprises at least one of the following properties:
VI) a viscose elasticity [tanδ (ω=0.3 rad/s)] in the range from about 0.1 to about 10;
V2) a liquid absorption of at least about 5 g/100 cm²;
V3) a water vapor permeability of at least about 100 g/(m²×24h); or
V4) an O₂ permeability of at least about 100 cm³/(m²×24h).

39. A composite according to claim 37, further comprising a film.

40. A composite according to claim 38, further comprising a film.

41. A composite according to claim 39, wherein the film has a water vapor permeability in the range from about 100 to about 2000 g/(m²×24h).

42. A composite according to claim 40, wherein the film has a water vapor permeability in the range from about 100 to about 2000 g/(m²×24h).

43. A composite according to claim 39, wherein the composition is directly adjacent to a film.

44. A composite according to claim 40, wherein the composition is directly adjacent to a film.

45. Active substance-doped water-absorbing polymer particles according to claim 22 comprising a hygiene article.

46. A water-absorbing composition according to claim 31 comprising a hygiene article.

47. A composite according to claim 37 comprising a hygiene article.

48. A process for producing a water-absorbing composition, the process comprising the step of:
a. providing a particulate water-absorbing polymer comprising an active substance;
b. forming a condensate matrix based on at least one polycondensate monomer; and
c. at least partially incorporating the particulate water-absorbing polymer into the condensate matrix,
wherein the particulate water-absorbing polymer comprises the active substance or
wherein an active substance-doped water-absorbing polymer particle comprising:
Φ1. an active substance in a quantity in the range from about 0.001 to about 30 wt. %, based on the active substance-doped water-absorbing polymer particles; and
Φ2. an absorber matrix in a quantity in the range from about 70 to about 99.999 wt. %, based on the active substance-doped water-absorbing polymer particles,
wherein the absorber matrix comprises a cross-linked polyacrylic acid to at least about 90 wt. %, based on the absorber matrix; and
wherein the cross-linked polyacrylic acid comprises, to at least about 90 wt. %, based on the cross-linked polyacrylic acid, an acrylic acid that is partially neutralized to at least about 30 mol. %, is contacted with the polycondensate monomer before the end of the polycondensate matrix formation.

49. A process according to claim 48, wherein the active substance comprises any one of a care substance, a wound-treating substance, or a care substance and a wound-treating substance.

50. A water absorbent composition obtainable by the process according to claim 48.

51. A water absorbent composition according to claim 50, wherein the water-absorbing polymer has at least one of the following properties:
A1) a particle size distribution, whereby at least about 80 wt. % of the particles have a particle size in a range from about 20 μm to about 900 μm according to ERT 420.1-99;
A2) a Centrifuge Retention Capacity (CRC) of at least about 10 g/g according to ERT 441.1-99;
A3) an Absorption Against Pressure (AAP) at about 0.7 psi of at least about 4 g/g according to ERT 442.1-99;
A4) a water soluble polymer content after about 16 hours extraction of less than about 25 wt. %, based on the total weight of the water-absorbing polymer, according to ERT 470.1-99; or A5) a residual moisture of at most about 15 wt. %, based on the total weight of the water-absorbing polymer, according to ERT 430.1-99.

52. A water absorbent composition according to claim 50, wherein the water-absorbing polymer comprises:
(α1) about 0.1 to about 99.999 wt. % polymerized, ethylenically unsaturated, acidic group-containing monomers or salts thereof or polymerized, ethylenically unsaturated monomers comprising a protonated or quaternated nitrogen, or mixtures thereof,
(α2) 0 to about 70 wt. % polymerized, ethylenically unsaturated monomers copolymerizable with (α1),
(α3) about 0.001 to about 10 wt. % of one or more crosslinkers,
(α4) 0 to about 30 wt. % water soluble polymers, and
(α5) 0 to about 20 wt. % of one or more auxiliaries,
wherein the sum of the weight quantities (α1) to (α5) amounts to about 100 wt. %.

53. A water absorbent composition according to claim 50, wherein the polycondensate matrix comprises at least about 10 wt. %, based on the polycondensate matrix, of a polyurethane.

54. A water absorbent composition according to claim 50, wherein the polycondensate matrix comprises a foam.

55. A composite comprising a water absorbent composition according to claim 50.

56. A composite according to claim 55, with at least one of the following properties:
VI) a viscose elasticity [tanδ (ω=0.3 rad/s)] in the range from about 0.1 to about 10;
V2) a liquid absorption of at least about 5 g/100 cm²;
V3) a water vapor permeability of at least about 100 g/(m²×24h); or V4) an O₂ permeability of at least about 100 cm³/(m²×24h).

57. A composite according to claim 55, further comprising a film.

58. A composite according to claim 57, wherein the film has a water vapor permeability in the range from about 100 to about 2000 g/(m²×24h).

59. A composite according to claim 57, wherein a water absorbent composition is directly adjacent to the film.

60. A water absorbent composition according to claim 50 comprising a hygiene article.

61. A composite according to claim 55 comprising a hygiene article.

62. A composite according to claim 60 comprising a hygiene article.

63. A method comprising using a composition according to claim 32 to release a wound-treating substance.

64. A method comprising using a composition according to claim 50 to release a wound-treating substance.

65. A method comprising using a water-absorbing polymer to release a wound-treating substance from a polycondensate matrix.

66. A method comprising using active substance-doped water-absorbing polymer particles according to claim 32 to treat a wound of a higher vertebrate organism or for preventing the formation of a wound at or in a higher vertebrate organism.

67. A method comprising using a composition according to claim 50 to treat a wound of a higher vertebrate organism or for preventing the formation of a wound at or in a higher vertebrate organism.

68. A method comprising using a composite according to claim 55 to treat a wound of a higher vertebrate organism or for preventing the formation of a wound at or in a higher vertebrate organism.

69. Using any one of:
(a) active substance-doped water-absorbing polymer particles comprising:
Φ1. an active substance in a quantity in the range from about 0.001 to about 30 wt. %, based on the active substance-doped water-absorbing polymer particles; and
Φ2. an absorber matrix in a quantity in the range from about 70 to about 99.999 wt. %, based on the active substance-doped water-absorbing polymer particles,
wherein the absorber matrix comprises a cross-linked polyacrylic acid to at least about 90 wt. %, based on the absorber matrix; and
wherein the cross-linked polyacrylic acid comprises, to at least about 90 wt. %, based on the cross-linked polyacrylic acid, an acrylic acid that is partially neutralized to at least about 30 mol. %;
(b) a water-absorbing composition comprising:
Γ1. a polycondensate matrix based on at least one polycondensate monomer with at least one polycondensate group; and
Γ2. a particulate water-absorbing polymer comprising an active substance including at least one functional group that can react with at least one polycondensate group to form a covalent link; or a particulate water-absorbing polymer comprising:
Φ1. an active substance in a quantity in the range from about 0.001 to about 30 wt. %, based on the active substance-doped water-absorbing polymer particles; and
Φ2. an absorber matrix in a quantity in the range from about 70 to about 99.999 wt. %, based on the active substance-doped water-absorbing polymer particles,
wherein the absorber matrix comprises a cross-linked polyacrylic acid to at least about 90 wt. %, based on the absorber matrix; and
wherein the cross-linked polyacrylic acid comprises, to at least about 90 wt. %, based on the cross-linked polyacrylic acid, an acrylic acid that is partially neutralized to at least about 30 mol. %,
wherein the particulate water-absorbing polymer is at least partially surrounded by the polycondensate matrix;
wherein at least the particulate water-absorbing polymer comprises the active substance; and
wherein the water-absorbing composition has an active substance availability of at least about 10 wt. % according to the Extraction Test described herein;
(c) a composite comprising a water-absorbing composition according to (b); or
(d) at least two thereof in a hygiene article or a wound treatment article.
